(19) United States
(12) Patent Application Publication
(10) Pub. No.: US 2025/0332126 A1
(43) Pub. Date: Oct. 30, 2025

(54) TOPICAL COMPOSITION FOR PAIN RELIEF

(71) Applicant: JOHNSON & JOHNSON CONSUMER INC., Skillman, NJ (US)

(72) Inventor: Kathleen Spisak, Delran, NJ (US)

(21) Appl. No.: 19/100,983

(22) PCT Filed: Aug. 21, 2023
(86) PCT No.: PCT/IB2023/058329

§ 371 Date: Feb. 4, 2025

Related U.S. Application Data
(60) Provisional application No. 63/373,260, filed on Aug. 23, 2022.

(51) Int. Cl. A61K 31/167; A61K 9/00; A61K 31/01; A61K 36/899; A61P 29/00

(52) U.S. Cl. CPC A61K 31/167; A61K 9/0014; A61K 31/01; A61K 36/899; A61P 29/00

(57) ABSTRACT

The present invention provides a topical composition for pain relief containing a topical analgesic, as well as a volatile emollient and/or an absorbant. The topical composition is substantially free of volatile alcohol. The topical composition may be used to relieve pain in a user with sensitive skin. Additionally, the present invention includes methods of relieving pain in a user with sensitive skin. The methods include topically applying to an exterior skin portion of the user's body in need of pain treatment a composition including a topical analgesic, as well as a volatile emollient and/or an absorbant.

### FIELD OF THE INVENTION

The present invention relates to topical compositions for pain relief; in particular, for pain relief of users with sensitive skin. More specifically, the present invention relates to a topical composition including a topical analgesic, as well as a volatile emollient and/or an absorbant. The topical composition is substantially free of volatile alcohol. Additionally, the present invention relates to methods of relieving pain, especially in users with sensitive skin. In particular, such methods include topically applying to an exterior skin portion of a user's body a composition including a topical analgesic, as well as a volatile emollient and/or an absorbant.

### BACKGROUND OF THE INVENTION

“Sensitive skin” is defined as the skin of any part of the body that has discomfort at any given time. Examples of conditions causing sensitive skin may include, but are not limited to, acne, contact dermatitis, dry skin, itchy skin and eczema. Sensitive skin may be described as skin that is “reactive” to external stimuli or that presents a special need. Examples of external stimuli may include sunlight, wind, water, air temperature, air moisture, skin and body hydration, diet and/or contact with chemical or physical materials.

Consumers with sensitive skin often find it difficult to find topical cosmetic or medicinal products that do not irritate or further irritate their skin. This is particularly detrimental to sensitive skin consumers who have health or cosmetic concerns (i.e., concerns unrelated to their sensitive skin) that are commonly addressed by topical skin treatments. One such example may be sensitive skin consumers who also suffer from body pain, for example pain in the neck, forearm, elbow, upper arm, wrist, calf, thigh, knee, back, shoulder, hip and/or ankle.

Pain is a common experience that may be of short duration (called “acute pain”) or of longer duration, for example, six months or longer (called “chronic pain”). Pain may negatively impact a person's quality of life and treatment of pain may be costly. Pain treatment options that are effective and inexpensive are desirable.

Body pain is often treated with a topical pain cream. Topical pain creams currently exist in various forms on the consumer market. However, such creams often provide a harsh user experience causing dry, red, irritated skin, an overly strong cooling or warming sensation and/or an overwhelming scent. Such creams are unsuitable for consumers with sensitive skin.

As such, there exists a need for topical pain compositions that provide a milder experience for users or consumers with sensitive skin. Such topical compositions could be used, for example as daily treatment, leave-on products.

### SUMMARY OF THE INVENTION

According to an example, a topical composition for pain relief may include a topical analgesic and a volatile emollient; where the topical composition is substantially free of volatile alcohol. Such topical composition may be suitable for users with sensitive skin.

According to an example, the topical analgesic may be in an amount of 0.5% to 8% by weight. According to an example, the topical analgesic may be one or more of lidocaine or benzocaine. According to a further example, the topical analgesic may be lidocaine, for example, in an amount of about 4% by weight.

According to an example, the volatile emollient may be in an amount of 1% to 12% by weight. According to a further example, the volatile emollient may be in an amount between about 4% to about 8% by weight. According to an example, the volatile emollient may provide a topical cooling sensation. According to a further example, the volatile emollient may be isododecane.

According to an example, the topical composition may further include an absorbant. According to an example, the absorbant may be in an amount of 0.1% to 5% by weight. According to a further example, the absorbant may be a processed oat ingredient. According to an example, the processed oat ingredient may be colloidal oat flour. According to an example, the colloidal oat flour may be in an amount of about 1% by weight.

According to an example, the topical composition may be substantially free of additional cooling agents, like menthol and menthol lactate.

According to an example, a method of relieving pain in a user with sensitive skin may include the step of applying a topical composition to an exterior skin portion of the user's body in need of pain treatment. The topical composition may include a topical analgesic and a volatile emollient; and the topical composition may be substantially free of volatile alcohol.

According to another example, a method of relieving pain in a user with sensitive skin may include topically applying to an exterior skin portion of a user's body in need of pain treatment a composition comprising lidocaine and isododecane; where the composition is substantially free of volatile alcohol. According to an example, the lidocaine may be in an amount of 0.5% to 8% by weight, and the isododecane may be in an amount of 1% to 12% by weight. According to a further example, the composition may further include a processed oat ingredient. According to an example, the processed oat ingredient may be in an amount of 0.1% to 5% by weight.

According to an alternative example, a topical composition may include a topical analgesic and an absorbant; where the topical composition is substantially free of volatile alcohol.

According to an example, the topical analgesic may be in an amount of 0.5% to 8% by weight. According to an example, the topical analgesic may be one or more of lidocaine or benzocaine. According to a further example, the topical analgesic may be lidocaine. According to an example, the lidocaine may be in an amount of about 4% by weight.

According to an example, the absorbant may be in an amount of 0.1% to 5% by weight. According to an example, the absorbant may be a processed oat ingredient. According to an example, the processed oat ingredient may be colloidal oat flour. According to an example, the colloidal oat flour may be in an amount of about 1% by weight.

According to an example, the topical composition may be substantially free of additional cooling agents, like menthol and menthol lactate.

According to one example, a method of relieving pain in a user with sensitive skin, includes the step of applying a topical composition to an exterior skin portion of the user's body in need of pain treatment. The topical composition may include a topical analgesic and an absorbant, where the topical composition is substantially free of volatile alcohol.

According to an example, a method of relieving pain in a user with sensitive skin includes topically applying to an exterior skin portion of the user's body in need of pain treatment a composition including lidocaine and colloidal oat flour, where the composition is substantially free of volatile alcohol. According to one example, the lidocaine may be in an amount of 0.5 to 8 wt % and the colloidal oat flour may be in an amount of 0.1 to 5 wt %.

### DETAILED DESCRIPTION

It is believed that one skilled in the art can, based on the description herein, utilize the present invention to its fullest extent. The following specific examples are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %”, “wt %”, “wt. %,” “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a “product” is optionally in finished packaged form. In one example, the package is a container such as a plastic, metal or glass tube or jar containing the composition. In one example, the composition may be squeezed or pumped out of the container. In another example, the composition may be applied to the skin using an applicator, such as a roller, roller ball(s), brush, a massage element or other functional applicator. According to a further example, the composition may not be suitable for a spray or aerosol dispensing system. The product may further contain additional packaging such as a plastic or cardboard box for storing such container. In one example, the product includes a composition of the invention and contains instructions directing the user to apply the composition to the skin.

As used herein, “topically applying” means directly laying on or spreading on outer skin, by use of the hands or an applicator such as a wipe, roller, or spray.

As used herein, “topical composition” means a composition that is applied to the outer skin of a user.

As used herein, “pain relief” refers to the alleviation of pain at one or more locations of the body.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular carrier utilized, and like factors.

As used herein, the term “about” refers to within 5% weight, within 4% weight, within 3% weight, within 2.5% weight, within 2% weight, or within 1% weight of a disclosed value.

One aspect of the invention pertains to a topical composition for pain relief that is suitable for users or consumers with sensitive skin. The topical composition includes a topical analgesic, as well as a volatile emollient and/or an absorbant; and the topical composition is substantially free of volatile alcohol.

In particular, it has been surprisingly discovered that the volatile emollient, and particularly isododecane, is sufficient to provide a cooling sensation gentle enough for sensitive skin users afflicted with body pain. Accordingly, another aspect of the invention pertains to a method of providing a cooling sensation to the sensitive skin of users afflicted with body pain, the method comprising applying to sensitive skin a composition comprising a topical analgesic and isododecane.

Additionally, it has been surprisingly discovered that the absorbant, and particularly colloidal oat flour (also known as Avena Sativa kernel flour), provides a gentle experience for sensitive skin users afflicted with body pain. Accordingly, another aspect of the invention pertains to a method of providing gentle pain relief to users afflicted with both sensitive skin and body pain, the method comprising applying to sensitive skin a composition comprising a topical analgesic and colloidal oat flour.

As used herein, “analgesic” means a class of drugs designed to relieve pain without causing the loss of consciousness. Such analgesics may be over-the-counter (OTC) or prescription drugs. Examples of analgesics may include, for example, acetaminophen, aspirin, COX inhibitors, and Nonsteroidal anti-inflammatory drugs (NSAIDs).

A “topical analgesic” refers to a drug, medication or pain-relieving agent that is applied on the skin to relieve muscle, joint or nerve pain. Topical analgesics may also be classified as counterirritants or anti-inflammatory agents in certain cases. Topical analgesics may be absorbed by the skin and may act on the tissue beneath. Examples of suitable topical analgesics may include lidocaine, capsaicin, nonsteroidal anti-inflammatory drugs (NSAIDs), salicylate rubefacients, benzocaine, tetracaine, nitroglycerin, rubefacients, such as camphor, menthol, methyl nicotinate and methyl salicylate, and salicylates, such as aspirin, magnesium salicylate and sodium salicylate. Capsaicin, menthol, methyl salicylate and camphor, for example, may also be classified as counter-irritants. A topical analgesic may include a combination of those listed above.

According to an example, the analgesic or topical analgesic may be recommended for external application to the skin, also referred to as the “exterior skin portion” of a user's body. For example, the topical analgesic should not be applied to internal body surfaces, such as the oral cavity. For this reason, the term “external analgesic” may be used simultaneously or in lieu of “topical analgesic.”

The analgesic or topical analgesic may be present in amounts ranging from about 0.1, 0.5, 1, 1.5, 2, 2.5, 3.1, 3.2, 3.3, 3.4, 3.5, 3.6, 3.7, 3.8, 3.9, 4, 4.5, or 5 to about 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 10 wt. % of the total composition. In one or more examples, the analgesic or topical analgesic is present in an amount ranging from about 0.1 wt. % to about 10 wt. % by total weight of the composition. In some examples, the analgesic or topical analgesic is present in an amount ranging from about 0.5 wt. % to about 8 wt. % by total weight of the composition. In one or more examples, the analgesic or topical analgesic is present in an amount ranging from about 2 wt. % to about 6 wt. % by total weight of the composition. In some examples, the analgesic or topical analgesic is present in an amount ranging from about 3 wt. % to about 5 wt. % by total weight of the composition. In one or more examples, the analgesic or topical analgesic is present in an amount ranging from about 3.5 wt. % to about 4.5 wt. % by total weight of the composition. In some examples, the analgesic or topical analgesic is present in an amount of about 4 wt. % by total weight of the composition.

In one or more examples, the topical analgesic comprises lidocaine, and is present in an amount ranging from about 0.1 wt. % to about 10 wt. % by total weight of the composition. In some examples, the lidocaine is present in an amount ranging from about 0.5 wt. % to about 8 wt. % by total weight of the composition. In one or more examples, the lidocaine is present in an amount ranging from about 2 wt. % to about 6 wt. % by total weight of the composition. In some examples, the lidocaine is present in an amount ranging from about 3 wt. % to about 5 wt. % by total weight of the composition. In one or more examples, the lidocaine is present in an amount ranging from about 3.5 wt. % to about 4.5 wt. % by total weight of the composition. In some examples, the lidocaine is present in an amount of about 4 wt. % by total weight of the composition.

As used herein, a “volatile emollient” refers to an emollient that has a flash point within 15° C. of the temperature of skin, and therefore does not persist on the skin after application. The volatile emollient may act as a cooling agent to provide a cooling sensation to the user's skin and will readily vaporize. Although not wishing to be bound to any particular theory, it is thought that the volatile emollient provides a cooling sensation on the skin solely due to evaporation of the ingredient from skin. It is thought there is a higher efficacy of cooling when it takes longer for ingredient to evaporate. According to one or more examples, the composition may include a volatile emollient, such as, for example, isododecane. Other examples of volatile emollients may include cyclopentasiloxane, dimethicone, isoparaffin cuts (C9-C16), ethyl trisiloxane, trisiloxane and caprylyl methicone. In some examples, the volatile emollient comprises isododecane.

The volatile emollient may be present in amounts ranging from about 0.1, 0.5, 1, 1.5, 2, 2.5, 3.1, 3.2, 3.3, 3.4, 3.5, 3.6, 3.7, 3.8, 3.9, 4.1, 4.2, 4.3, 4.4, 4.5, 5, or 6 to about 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 10, 11, 12 wt. % of the total composition. In another example, the volatile emollient may be present in amounts ranging from about 0.1, 0.5, 1, 2, 2.5, 3, 3.5, 4, 4.5, 5, 5.5, 6, 6.5, 7, 7.1, 7.2, 7.3, 7.4, 7.5, 7.6, 7.7, 7.8, 7.9, 8, 8.1, 8.2, 8.3, 8.4, 8.5, or 9 to about 7.5, 7.6, 7.7, 7.8, 7.9, 8, 8.1, 8.2, 8.3, 8.4, 8.5, 8.6, 8.7, 8.8, 8.9, 9, 9.5, 10, 11, 12 wt. % of the total composition. In one or more examples, the volatile emollient is present in an amount ranging from about 0.1 wt. % to about 12 wt. % by total weight of the composition. In some examples, the volatile emollient is present in an amount ranging from about 1 wt. % to about 12 wt. % by total weight of the composition. In one or more examples, the volatile emollient is present in an amount ranging from about 2 wt. % to about 10 wt. % by total weight of the composition. In some examples, the volatile emollient is present in an amount ranging from about 3 wt. % to about 9 wt. % by total weight of the composition. In one or more examples, the volatile emollient is present in an amount ranging from about 3.5 wt. % to about 8.5 wt. % by total weight of the composition. In some examples, the volatile emollient is present in an amount ranging from about 4 wt. % to about 8 wt. % by total weight of the composition. In one or more examples, the volatile emollient is present in an amount of about 4 wt. % by total weight of the composition. In some examples, the volatile emollient is present in an amount of about 8 wt. % by total weight of the composition.

In one or more examples, the volatile emollient comprises isododecane. The isododecane may be present in amounts ranging from about 0.1, 0.5, 1, 1.5, 2, 2.5, 3.1, 3.2, 3.3, 3.4, 3.5, 3.6, 3.7, 3.8, 3.9, 4.1, 4.2, 4.3, 4.4, 4.5, 5, or 6 to about 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 10, 11, 12 wt. % of the total composition. In another example, the isododecane may be present in amounts ranging from about 0.1, 0.5, 1, 2, 2.5, 3, 3.5, 4, 4.5, 5, 5.5, 6, 6.5, 7, 7.1, 7.2, 7.3, 7.4, 7.5, 7.6, 7.7, 7.8, 7.9, 8, 8.1, 8.2, 8.3, 8.4, 8.5, or 9 to about 7.5, 7.6, 7.7, 7.8, 7.9, 8, 8.1, 8.2, 8.3, 8.4, 8.5, 8.6, 8.7, 8.8, 8.9, 9, 9.5, 10, 11, 12 wt. % of the total composition. In one or more examples, the isododecane is present in an amount ranging from about 0.1 wt. % to about 12 wt. % by total weight of the composition. In some examples, the isododecane is present in an amount ranging from about 1 wt. % to about 12 wt. % by total weight of the composition. In one or more examples, the isododecane is present in an amount ranging from about 2 wt. % to about 10 wt. % by total weight of the composition. In some examples, the isododecane is present in an amount ranging from about 3 wt. % to about 9 wt. % by total weight of the composition. In one or more examples, the isododecane is present in an amount ranging from about 3.5 wt. % to about 8.5 wt. % by total weight of the composition. In some examples, the isododecane is present in an amount ranging from about 4 wt. % to about 8 wt. % by total weight of the composition. In one or more examples, the isododecane is present in an amount of about 4 wt. % by total weight of the composition. In some examples, the isododecane is present in an amount of about 8 wt. % by total weight of the composition.

As used herein, an “absorbant” is an ingredient that has the capacity to absorb or soak up liquids. According to an example, the absorbant may be a “processed oat ingredient,” which refers to an ingredient that is typically derived from a part of the oat plant (Avena sativa). Processed oat ingredients are known to have antioxidant, antifungal and anti-inflammatory properties. Processed oat ingredients have been shown to be effective in treating sensitive skin, including skin that is dry, rough, flaky and itchy. In a powdery format, processed oat ingredients can also absorb oils and may be beneficial for those with oily skin.

The processed oat ingredient can be either a processing (e.g., an extract, milling, fermenting) of one or more parts of the oat plant (e.g., grain, leaf, stem, seed) or can be a molecule found in the oat plant (e.g., beta-glucan, flavonoids, avenanthramides, lipids, peptides, etc.). This definition is intended to cover processed oat ingredients which are derived from other sources other than oat (e.g., from another plant or chemically synthesized), but are otherwise associated with oat. In one or more examples, the processed oat ingredient is selected from the group consisting of oat extract, colloidal oatmeal, colloidal oat flour, oat flour, oat bran, oat protein, oat peptide, oat oil, fermented oat, avenanthramides, beta-glucan, modified oat grain material (e.g., chemically, enzymatically-, microorganism-modified), and combinations thereof.

As used herein, “colloidal oat flour” or “colloidal oatmeal” means the powder resulting from the grinding and further processing of whole oat grain meeting United States Standards for Number 1 or Number 2 oats. The colloidal oat flour has a particle size distribution as follows: not more than 3 percent of the total particles exceed 150 micrometers in size and not more than 20 percent of the total particles exceed 75 micrometers in size. Examples of suitable colloidal oat flours include, but are not limited to, “Tech-0” available from the Beacon Corporation and colloidal oat flour available from Quaker.

The processed oat ingredient(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition. In one or more examples, the processed oat ingredient(s) is present in an amount ranging from about 0.1 wt. % to about 10 wt. % by total weight of the composition. In some examples, the processed oat ingredient(s) is present in an amount ranging from about 0.1 wt. % to about 5 wt. % by total weight of the composition. In one or more examples, the processed oat ingredient(s) is present in an amount ranging from about 0.5 wt. % to about 5 wt. % by total weight of the composition. In some examples, the processed oat ingredient(s) is present in an amount ranging from about 0.5 wt. % to about 4 wt. % by total weight of the composition. In one or more examples, the processed oat ingredient(s) is present in an amount ranging from about 0.5 wt. % to about 3 wt. % by total weight of the composition. In some examples, the processed oat ingredient(s) is present in an amount ranging from about 0.5 wt. % to about 2.5 wt. % by total weight of the composition. In one or more examples, the processed oat ingredient(s) is present in an amount of about 0.5 wt. % to about 1.5 wt. % by total weight of the composition. In some examples, the processed oat ingredient(s) is present in an amount of about 1 wt. % by total weight of the composition.

In one or more examples, the processed oat ingredient comprises colloidal oat flour, and present in an amount ranging from about 0.1 wt. % to about 10 wt. % by total weight of the composition. In some examples, the colloidal oat flour is present in an amount ranging from about 0.1 wt. % to about 5 wt. % by total weight of the composition. In one or more examples, the colloidal oat flour is present in an amount ranging from about 0.5 wt. % to about 5 wt. % by total weight of the composition. In some examples, the colloidal oat flour is present in an amount ranging from about 0.5 wt. % to about 4 wt. % by total weight of the composition. In one or more examples, the colloidal oat flour is present in an amount ranging from about 0.5 wt. % to about 3 wt. % by total weight of the composition. In some examples, the colloidal oat flour is present in an amount ranging from about 0.5 wt. % to about 2.5 wt. % by total weight of the composition. In one or more examples, the colloidal oat flour is present in an amount of about 0.5 wt. % to about 1.5 wt. % by total weight of the composition. In some examples, the colloidal oat flour is present in an amount of about 1 wt. % by total weight of the composition.

According to an example, the composition may also exclude certain ingredients. The term “substantially free of” refers to a composition having less than 5% by weight, less than 4% by weight, less than 3% by weight, less than 2% by weight, less than 1% by weight, less than 0.75% by weight, or less than 0.5% by weight of a particular ingredient. The term “completely free of” refers to a composition having less than 0.5% by weight, less than 0.4% by weight, less than 0.3% by weight, less than 0.2% by weight, less than 0.1% by weight or the complete absence of an ingredient.

According to an example, the composition may be substantially free of volatile alcohol. The term “volatile alcohol” refers to an alcohol which will readily vaporize, or an alcohol with a vapor pressure which is higher than that of water at 20° C. According to another example, the composition may be completely free of volatile alcohol. Examples of volatile alcohols include, but are not limited to, specially denatured (SD) alcohol, for example SD Alcohol 40, denatured alcohol, ethanol, methanol and isopropyl alcohol. The vapor pressure of various alcohols at 20° C. include ethanol with a vapor pressure of 5.95 kPa (kilopascals), methanol with a vapor pressure of 11.9 kPa and isopropanol with a vapor pressure of 4.40 kPa.

According to an example, the composition may be substantially free of or completely free of cooling agents such as menthol, or cooling sensates such as menthyl esters like menthol lactate. Other examples of cooling agents may include menthyl glutarate, N-ethyl 2-isopropyl-5-methylcyclohexanecarboxamide, N-[(ethoxycarbonyl)methyl)-p-menthane-3-carboxamide and (1R,2S,5R)-N-(4-methoxyphenyl)-5-methyl-2-(1-methylethyl)cyclohexanecarboxamide.

Cooling agents may include a phenyl ring and a polar side group, for example Menthyl Glutarate. In certain examples a combination of more than one cooler comprising a phenyl ring with a polar side group may be used as a cooling agent. In one example a combination of a cooler comprising a phenyl ring with a polar side group and a cooler without a phenyl ring with a polar side group may be used as a cooling agent. In another example, the cooler comprising a phenyl ring with a polar side group is a cooler with a cyclohexyl core substituted at ring positions 1, 3 and 4 and an ester or an amide group at one of the three positions.

The composition may include one or more “thickeners,” or “viscosity increasing agents,” selected from, for example, ammonium acrylolydimethyltaurate/vinylpyrrolidone copolymer, sodium acrylolydimethyltaurate/vinylpyrrolidone copolymer, sodium polyacrylate, carbomer, and/or acrylates copolymer. According to an example, the composition may include about 0.1 to about 10, or about 0.5 to about 5, or about 1 to 4 weight % thickener. The amount and selection of the thickener allows for stabilization of the cream or gel cream while maintaining the cream or gel cream aesthetic and skin feel.

In an example, the composition may include about 0.1 to about 0.8 weight % carbomer and about 0.5 to about 1.0 weight % sodium polyacrylate. The composition may, for example, include about 0.4 weight % carbomer and about 0.75 weight % sodium polyacrylate.

In another example, the composition may include about 0.1 to about 0.8 weight % carbomer, about 0.4 to about 1.0 weight % sodium polyacrylate and about 1.0 to about 3.0 weight % acrylates copolymer. The composition may, for example, include about 0.4 weight % carbomer, about 0.6 weight % sodium polyacrylate and about 2.0 weight % acrylates copolymer.

As used herein, the term “emulsifier” refers to an ingredient that helps keep ingredients (such as oil and water) from separating in an emulsion. According to an example, an example of an emulsifier may include cetyl alcohol. According to an example, the composition may include about 0.1 to about 10, or about 1 to about 6, or about 2.5 to 4.5 weight % emulsifier. In one example, the emulsifier may be about 3 wt. %,

The compositions of the present invention are applied topically to human skin. Accordingly, the composition may further include cosmetically acceptable topical ingredients as known in the personal care art for use with cream formulations or gel-cream formulations of the oil-in-water emulsion type.

The composition may contain for example suitable gelling agents such as natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain between about 0.1% and 5%, by weight, of such gelling agents.

The compositions of the present invention may further include any of a variety of additional cosmetically active agents. Examples of suitable additional active agents may include: anti-microbial, anti-fungal, and anti-bacterial agents, anti-inflammatory agents, additional external analgesics, sunscreens, photoprotectors, antioxidants, moisturizers, nutrients, vitamins, energy enhancers, hydration boosters, efficacy boosters, agents for skin conditioning, odor-control agents such as odor masking or pH-changing agents, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred examples, in an amount of 0.1% to 5% and in other preferred examples from 1% to 2%.

As used herein, a “skin conditioning agent,” or “skin conditioner” generally refers to an ingredient that enhances the appearance of dry or damaged skin by reducing flaking and/or restoring suppleness. For example, a skin conditioning agent may act as a lubricant on the skin surface to give the skin a soft and smooth appearance. Or, a skin conditioning agent may increase the water content of the top layer of the skin by drawing moisture from the surrounding air. In certain examples, the composition may include at least one skin conditioning agent. In one example, the composition may include zingiber officinale (ginger) root extract, caprylyl glycol, 1,2-hexanediol and/or tropolone.

Compositions of the present invention may include a cosmetically effective amount of one or more skin conditioning agents such as those described above. The compositions preferably include, on an active basis, from about 0.1% to about 10% by weight of the skin conditioning agents, more preferably from about 0.5% to about 5% of skin conditioning agents, and most preferably from about 0.5% to about 2% of skin conditioning agents.

In one example, the composition may include caprylyl glycol in an amount from about 0.1% to about 0.5% by weight and a combination of 1,2-hexanediol, capryl glycol and tropolone in an amount from about 0.1% to about 1.0% by weight. The composition may include caprylyl glycol in an amount of about 0.26% by weight and a combination of 1,2-hexanediol, capryl glycol and tropolone in an amount of about 0.5% by weight. The composition may further include zingiber officinale (ginger) root extract in an amount from about 0.1% to about 0.3% by weight, for example about 0.2% by weight.

Compositions of the present invention may include a cosmetically effective amount of one or more anti-inflammatory compounds. Examples of suitable anti-inflammatory agents may include substituted resorcinols, (E)-3-(4-methylphenylsulfonyl)-2-propenenitrile (such as “Bay 11-7082,” commercially available from Sigma-Aldrich of St. Louis, Missouri), tetrahydrocurcuminoids (such as Tetrahydrocurcuminoid CG, available from Sabinsa Corporation of Piscataway, NJ), extracts and materials derived from the following: Phellodendron amurense Cortex Extract (PCE), Non-Denatured Soy (Glycine max), Feverfew (Tanacetum parthenium), Ginger (Zingiber officinale), Ginko (Ginkgo biloba), Madecassoside (Centella asiatica extract ingredient), Cotinus (Cotinus coggygria), Butterbur Extract (Petasites hybridus), Goji Berry (Lycium barbarum), Milk Thistle Extract (Silybum marianum), Honeysuckle (Lonicera japonica), Basalm of Peru (Myroxylon pereirae), Sage (Salvia officinalis), Cranberry Extract (Vaccinium oxycoccos), Amaranth Oil (Amaranthus cruentus), Pomegranate (Punica granatum), Yerbe Mate (Ilex paraguariensis Leaf Extract), White Lily Flower Extract (Lilium candidum), Olive Leaf Extract (Oleaeuropaea), Phloretin (apple extract), Oat Flour (Aveena sativa), Lifenol (Hops: Humulus lupulus) Extract, Bugrane P (Ononis spinosa), Licochalcone (Licorice: Glycyrrhiza inflate extract ingredient), Symrelief (Bisabolol and Ginger extract), combinations of two or more thereof, and the like.

In certain examples the composition may include an anti-inflammatory compound in an amount from about 0.5% to about 2% by weight, and more preferably from about 0.5% to about 1.5%. In another example, the anti-inflammatory compound may include colloidal oat flour and/or ginger.

The pH of the composition may be adjusted using pH adjusting agents commonly used in the art. In one or more examples, the composition has a pH of from about 4.5 to about 5.5. In some examples, the pH of the composition ranges from about 3, 3.1, 3.2, 3.3, 3.4, 3.5, 3.6, 3.7, 3.8 or 3.9 to about 3.8, 3.9, 4, 4.1, 4.2, 4.3, 4.4, 4.5, 4.6, 4.7, 4.8, 4.9 or 5. In some examples, the pH of the composition ranges from about 3 to about 5, or about 3.5 to about 4.5. In other examples, the pH of the composition may range from about 6, 6.1, 6.2, 6.3, 6.4, 6.5, 6.6, 6.7, 6.8 or 6.9 to about 6.8, 6.9, 7, 7.1, 7.2, 7.3, 7.4, 7.5, 7.6, 7.7, 7.8, 7.9 or 8. In some examples, the pH of the composition ranges from about 6 to about 7.

In one example, the pH adjusting agent may be citric acid in an amount from about 0.1% to about 10% by weight, more preferably from about 0.5% to about 5%, and most preferably from about 0.5% to about 1% by weight. According to an example, the citric acid may be in an amount of about 0.6 wt. %.

A variety of other materials may optionally be present in the compositions of the present invention. In certain preferred examples, the composition includes one or more topical ingredients selected from the group consisting of: surfactants, chelating agents, additional emollients, humectants, conditioners, preservatives, opacifiers, fragrances and the like.

“Emollients” may include compounds that help to maintain the soft, smooth, and pliable appearance of the skin (e.g., by remaining on the skin surface or in the stratum corneum to act as a lubricant). Examples of suitable emollients include, but are not limited to, petrolatum, isopropyl palmitate, cetearyl olivate, sorbitan olivate, Euphorbia Cerifera (Candelila) wax, isododecane, hexyldecyl stearate and plant, nut, and vegetable oils and hydrogenated vegetable oils such as macadamia nut oil, rice bran oil, grape seed oil, palm oil, prim rose oil, hydrogenates peanut oil, and avocado oil. According to an example, the emollients may include isopropyl palmitate, cetearyl olivate, sorbitan olivate, Euphorbia Cerifera (Candelilla) Wax, hydrogenated vegetable oil, vegetable oil and combinations thereof.

In certain examples the composition may include emollients in an amount from about 0.1% to about 15% by weight, and more preferably from about 4% to about 11%, about 7% to about 10%, or about 8% to about 9% by weight. According to one example, the emollients are about 8.5 wt. %.

In one example, the composition may include isopropyl palmitate in an amount from about 4.0% to about 7.0% by weight, a combination of cetearyl olivate and sorbitan olivate in an amount from about 0.1% to about 1.0% by weight, cetyl alcohol in an amount from about 0.1% to about 5.0% by weight, and a combination of euphorbia cerifera (Candelilla) wax, hydrogenated vegetable oil and vegetable oil in an amount from about 1.0% to about 5.0% by weight. The composition may include isopropyl palmitate in an amount of about 5.0% by weight, a combination of cetearyl olivate and sorbitan olivate in an amount of about 0.5% by weight, cetyl alcohol in an amount of about 1.0% by weight, and a combination of euphorbia cerifera (Candelilla) wax, hydrogenated vegetable oil and vegetable oil in an amount of about 2.0% by weight. In another example, the composition may include isopropyl palmitate in an amount of about 5.0% by weight, a combination of cetearyl olivate and sorbitan olivate in an amount of about 0.5% by weight, cetyl alcohol in an amount of about 3.0% by weight, and a combination of euphorbia cerifera (Candelilla) wax, hydrogenated vegetable oil and vegetable oil in an amount of about 3.0% by weight.

In another example, the composition may further include isododecane as a volatile emollient in an amount of from about 1.0% to about 12.0% by weight, for example about 4.0% by weight or about 8.0% by weight. The composition may further exclude isododecane.

As used herein, “oils” means organic, hydrophobic compounds that are liquid at 25° C. excluding silicone-based materials. Examples of organic oils include various hydrocarbons (straight or branched chain alkanes or alkenes, ketone, diketone, primary or secondary alcohols, aldehydes, sterol esters, alkanoic acids, turpenes, monoesters), such as those having a carbon chain length ranging from C6-C38, such as C6-C18.

Specific non-limiting examples of oils include without limitation natural oils such as castor oil, mineral oil, trigylcerides, esters of an alcohol (glycerol or other than glycerol including diesters or other branched esters) and a fatty acid or fatty alcohol, and various natural waxes including shea (Butyrospermum parkii) butter, lotus wax; beeswax, insect waxes, sperm whale oil, lanolin, vegetable waxes such as canauba wax, jojoba oil, candelilla wax; mineral waxes such as paraffin wax; and synthetic waxes such as cetyl palmitate, lauryl palmitate, cetostearyl stearate, and polyethylene wax.

Oils may include for example esters such as isopropyl palmitate, isopropyl myristate, isononyl isonanoate (such as WICKENOL 151 available from Alzo Inc. of Sayreville, NJ), C12-C15 alkyl benzoates (such as FINSOLV TN from Innospec Active Chemicals), caprylic/capric triglycerides, pentaerythritol tetraoctanoate, mineral oil, dipropylene glycol dibenzoate, PPG-15 stearyl ether benzoate, PPG-2-Myristyl Ether Propionate, ethyl methicone, and diethylhexylcyclohexane. Further examples of oils include functional oils such as vitamin E acetate.

What is meant by a “humectant” is a compound intended to increase the water content of the top layers of skin (e.g., hygroscopic compounds). Examples of suitable humectants include, but are not limited to, glycerin, sorbitol or trehalose (e.g., α,α-trehalose, β,β-trehalose, α,β-trehalose) or a salt or ester thereof (e.g., trehalose 6-phosphate). In one example, the humectant is glycerin (also spelled “glycerine” interchangeably).

In certain examples the composition may include a humectant in an amount from about 1% to about 20% by weight, and more preferably from about 10% to about 15%. According to one example, the humectant is about 12 wt. %. According to another example, the humectant is about 14 wt. %.

As used herein, a “preservative” refers to an ingredient that may prevent or retard bacterial growth and thus protect a cosmetic product from spoilage. Suitable preservatives include, for example, caprylyl glycol, 1,2-hexanediol, tropolone and/or chlorphenesin and are present in the composition in an amount, based upon the total weight of the composition, from about 0 to about 5 percent or from about 0.05 percent to about 2 percent. According to one example, the preservative is present in an amount of about 0.56 wt. %.

In one example, the composition may include a preservative of caprylyl glycol in an amount from about 0.1% to about 0.5% by weight and chlorphenesin in an amount from about 0.1 to about 0.5% by weight. The composition may include caprylyl glycol in an amount of about 0.26% by weight and chlorphenesin in an amount of about 0.3% by weight.

Any of a variety of commercially available pearlescent or opacifying agents are suitable for use in the composition. Examples of suitable pearlescent or opacifying agents include, but are not limited to, mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)ₐ-H, wherein J is an alkylene group having from about 2 to about 3 carbon atoms; and a is 2 or 3; fatty alcohols containing from about 16 to about 22 carbon atoms; fatty esters of the formula: KCOOCH2L, wherein K and L independently contain from about 15 to about 21 carbon atoms; inorganic solids insoluble in the composition, and mixtures thereof.

Any fragrance compositions suitable for use on skin may be used in accordance with the present invention. A fragrance may be in the amount of 0.1% to 2% by weight.

According to an example, the composition may contain water, for example purified water, to serve as a vehicle. Water may be added from about 50% to about 95% by weight of the composition depending on the product form, as discussed below. Water may be added to achieve quantum satis (“q.s.” or “Q.S.”) in a formulation.

### Product Form and Packaging

A variety of product forms and packaging may be suitable for any of the compositions described herein. As noted earlier, a “product” is optionally in finished packaged form. In one example, the package is a container such as a plastic, metal or glass tube or jar containing the composition. In one or more examples, the composition may be squeezed or pumped out of the container. The product may further contain additional packaging such as a plastic or cardboard box for storing such container. In one or more examples, the product includes a composition of the invention and contains instructions directing the user to apply the composition to the skin.

Any of the compositions may be in the form of emulsions, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. Reference in the instant application is made to two types of viscosity measurements: A) “rheometer viscosity,” which is measured as a steady-state value at an applied shear rate of 20 s⁻¹ in a rheometer, at 25° C., and B) “Brookfield viscosity,” which is measured at 5 or 10 RPM after one minute at 25° C. in 4 oz jar using spindle RV #4 or RV #5. Unless otherwise specified, the viscosity referenced is the rheometer viscosity.

In one or more examples, the compositions described herein may be in the form of a lotion, gel, cream or gel-cream product. Lotion, gel, cream and gel-cream products may advantageously have the properties to allow for the compositions to be flowable and applied onto skin. Such products are generally thin enough to allow the volatile emollient to evaporate and provide the cooling effect. If too thick, as with an ointment, occlusive or patch, the user may not perceive a cooling affect.

In one or more examples, the compositions described herein are in the form of a lotion. As used herein, the term “lotion” means a predominantly water-containing topical preparation of light texture and fresh watery sensation, having a viscosity of from about 1,000 cPs to 4,000 cPs. Lotions or serums typically contain at least one emollient in addition to a solvent. Lotions/serums may comprise from about 1% to about 20% (e.g., from about 5% to about 10%) of an emollient(s) and from about 50% to about 90% (e.g., from about 60% to about 80%) of water.

In one or more examples, the compositions described herein are in the form of a cream. As used herein, the term “cream” means a predominantly water-containing topical preparation of rich texture having a viscosity of from about 2,000 cPs to 8,000 cPs. A cream typically contains from about 5% to about 50% (e.g., from about 10% to about 20%) of an emollient(s) and from about 45% to about 85% (e.g., from about 50% to about 75%) of water.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type, and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel. As used herein, the term “gel” means a predominantly water-containing topical preparation of rich texture containing dispersing polymer and exhibiting yield value of about 0.1 Pa or more. The gel may contain a gelling agent. Such gels typically contain between about 0.1% and 5%, by weight, of such gelling agents.

According to a further example, compositions of this invention may be formulated as a gel-cream. As used herein, the term “gel-cream” means a predominantly water-containing topical preparation of rich texture that has some user aesthetics of both a cream and a gel, as defined above. The gel-cream may have a viscosity of from about 2,000 cPs to 8,000 cPs. A gel-cream may contain from about 5% to about 50% (e.g., from about 10% to about 20%) of an emollient(s) and from about 45% to about 85% (e.g., from about 50% to about 75%) of water. A gel-cream may contain a dispersing polymer and exhibit a yield value of about 0.1 Pa or more. A gel-cream may contain one or more gelling agents, for example, between about 0.1% and 5% bv weight of such gelling agents.

The compositions of the present invention can also be formulated into a solid formulation (e.g., wipe). The composition of the present invention can also be combined with and/or impregnated on to a solid, semi-solid, or dissolvable substrate (e.g., a wipe, mask, pad, glove, or strip).

The compositions described herein can be provided to the consumer in a container, e.g., a bottle, tube, etc. Individual packettes enclosing measured portions of the composition may also be used. To dispense the composition from a bottle, a pump, squeezable valve, or a removable screw cap may be used.

### Methods

The present invention further includes a method of relieving pain by topically applying the composition of the invention to the skin in areas in need of pain relief. The method includes, for example, topically applying the composition to skin in a location of the body in need of pain relief. Such topical application may be to any skin in need of treatment on the body, for example skin of the face, neck, chest, back, hips, arms, axilla, hands, feet and/or legs.

The present invention includes a method of relieving pain by topically applying the composition of the invention to the skin in areas in need of pain relief, where the user or subject has sensitive skin. The method includes, for example, topically applying the composition to sensitive skin in a location of the body in need of pain relief. Such topical application may be to any skin in need of treatment on the body, for example skin of the face, neck, chest, back, hips, arms, axilla, hands, feet and/or legs.

The compositions may be applied to the skin as needed, or otherwise how often the user desires. In one or more examples, the composition is applied once or twice per day.

Any suitable method of applying the composition to the skin in need may be used. For example, the composition may be applied directly from a package to the skin in need, by hand to the skin in need, using an applicator to the skin in need, or may be transferred from a substrate such as a wipe or mask, or a combination of two or more thereof. In other examples, the composition may be applied via a dropper, tube, roller, pump, and patch or added to a bath or otherwise to water to be applied to the skin, and the like. The composition may be applied in a variety of manners/forms, including, without limitation, as a leave-on cream, a rinse-off cream, and/or as part of a patch.

The composition, and formulations and products containing such composition, may be prepared using methodology that is well known by an artisan of ordinary skill. The composition may be used on a routine basis and is substantially free of skin irritants.

### EXAMPLES

The following examples set forth below further illustrate examples of the present invention. All amounts are given in weight percent. The present invention is not limited to the examples contained therein.

### Example 1

Topical Gel-Cream with Isododecane

A topical gel-cream composition according to an example of the invention was made having the following ingredients, as shown in Table 1.

TABLE 1
Ingredient Listing for Example 1

| Ingredient | Primary Function | Weight % |
|---|---|---|
| Purified Water | Vehicle | Q.S. |
| Colloidal Oat Flour | Absorbant | 1.00 |
| Carbomer (Carbomer Ultrez 10 | Viscosity Increasing Agent | 0.40 |
| Polymer)¹ | | |
| Citric Acid Anhydrous USP | pH Adjusting Agent | 0.60 |
| Lidocaine USP | Pain Relieving Agent | 4.00 |
| Glycerine | Humectant | 14.00 |
| Sodium Polyacrylate² | Viscosity Increasing Agent | 0.75 |
| Isopropyl Palmitate³ | Emollient | 5.00 |
| Cetearyl Olivate; Sorbitan | Emollient | 0.50 |
| Olivate⁴ | | |
| Cetyl Alcohol⁵ | Emulsifier | 1.00 |
| Euphorbia Cerifera | Emollient | 2.00 |
| (Candelilla) Wax; | | |
| Hydrogenated Vegetable | | |
| Oil; Vegetable Oil⁶ | | |
| Isododecane⁷ | Emollient | 4.00 |
| Zingiber Officinale | Skin Conditioner | 0.20 |
| (Ginger) Root Extract | | |
| Caprylyl Glycol⁸ | Preservative System | 0.26 |
| | Component and | |
| | Skin Conditioner | |
| 1,2-Hexanediol; Caprylyl | Skin Conditioner | 0.50 |
| Glycol; Tropolone⁹ | | |
| Chlorphenesin¹⁰ | Preservative | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft VP from the UL Prospector Corporation
⁷Commercially available as Permethyl 99A from the Presperse Corporation
⁸Commercially available as Hydrolite CG from the Symrise Corporation
⁹Commercially available as Symdiol 68T from the Symrise Corporation
¹⁰Commercially available as Germazide C from the BASF Corporation

The following mixing procedure may be used to prepare Example 1:
a) The purified water is added to a suitable vessel and the colloidal oat flower is added while mixing.
b) The citric acid and lidocaine USP are added while mixing and mixed until dissolved.
c) The carbomer is added while mixing slowly until all clumps are dissolved.
d) The mixture is heated until 75-80°° C.
e) The caprylyl glycol and chlorphenesin are added while mixing and mixed until uniform.
f) The sodium polyacrylate is then added slowly while mixing at 75-80° C. until uniform.
g) The oil phase ingredients are then added while mixing (7% of the glycerin, isopropyl palmitate, cetearyl olivate; sorbitan olivate, and cetyl alcohol).
h) The mixture is mixed for 10 minutes and then homogenized for 3 minutes at 5000 RPM.
i) The mixture is cooled to 25° C. and additional 7% of glycerin and the isododecane is added and mixed.
j) The zingiber officinale root extract, 1,2-Hexanediol; Caprylyl Glycol; Tropolone are added and mixed until homogeneous.
k) At or below 35° C., the pH is adjusted to 6.5 using Sodium Hydroxide Solution or Citric Acid Solution and mixed.
l) The batch is brought to volume with water to 95-100% of theoretical batch weight.

### Example 2

Topical Cream with Isododecane

A topical cream composition according to an example of the invention was made having the following ingredients, as shown in Table 2.

TABLE 2
Ingredient Listing for Example 2

| Ingredient | Primary Function | Weight % |
|---|---|---|
| Purified Water | Vehicle | Q.S. |
| Colloidal Oat Flour | Absorbant | 1.00 |
| Carbomer (Carbomer Ultrez 10 | Viscosity Increasing Agent | 0.40 |
| Polymer)¹ | | |
| Citric Acid Anhydrous | pH Adjusting Agent | 0.60 |
| Lidocaine USP | Pain Relieving Agent | 4.00 |
| Glycerine | Humectant | 12.00 |
| Sodium Polyacrylate² | Viscosity Increasing Agent | 0.60 |
| Isopropyl Palmitate³ | Emollient | 5.00 |
| Cetearyl Olivate; Sorbitan | Emollient | 0.50 |
| Olivate⁴ | | |
| Cetyl Alcohol⁵ | Emulsifier | 3.00 |
| Euphorbia Cerifera | Emollient | 3.00 |
| (Candelilla) Wax; | | |
| Hydrogenated Vegetable | | |
| Oil; Vegetable Oil⁶ | | |
| Isododecane⁷ | Emollient | 8.00 |
| Fragrance | Fragrance | 0.50 |
| Acrylates Copolymer⁸ | Viscosity Increasing Agent | 2.00 |
| Caprylyl Glycol⁹ | Preservative System | 0.26 |
| | Component and | |
| | Skin Conditioner | |
| 1,2-Hexanediol; Caprylyl | Skin Conditioner | 0.50 |
| Glycol; Tropolone¹⁰ | | |
| Chlorphenesin¹¹ | Preservative | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft BP from the UL Prospector Corporation
⁷Commercially available as Permethyl 99A from the Presperse Corporation
⁸Commercially available as Carbopol Aqua SF-1 Polymer from the Lubrizol Corporation
⁹Commercially available as Hydrolite CG from the Symrise Corporation
¹⁰Commercially available as Symdiol 68T from the Symrise Corporation
¹¹Commercially available as Germazide C from the BASF Corporation

The following mixing procedure may be used to prepare Example 2:
a) The purified water is added to a suitable vessel and the colloidal oat flower is added while mixing.
b) The citric acid and lidocaine USP are added while mixing and mixed until dissolved.
c) The carbomer is added while mixing slowly until all clumps are dissolved.
d) The mixture is heated until 75-80°° C.
e) The caprylyl glycol and chlorphenesin are added while mixing and mixed until uniform.
f) The sodium polyacrylate is then added slowly while mixing at 75-80° C. until uniform.
g) The oil phase ingredients are then added while mixing (7% of glycerin, isopropyl palmitate, cetearyl olivate; sorbitan olivate, and cetyl alcohol).
h) The mixture is mixed for 10 minutes and then homogenized for 3 minutes at 5000 RPM.
i) The mixture is cooled to 25° C. and the isododecane is added and mixed.
j) The fragrance, 1,2-hexanediol; caprylyl glycol; tropolone are added and mixed until homogeneous.
k) At or below 35° C., the pH is adjusted to 6.5 using sodium hydroxide solution or citric acid solution and mixed.
l) In a different vessel 5% glycerin and acrylates Copolymer are added and mixed until uniform to create the acrylates copolymer premix.

m) The acrylates copolymer premix is added to the mixture from Step 11 and mixed until homogenous.

### Example 3

### Topical Gel-Cream Without Isododecane

According to an example the topical gel-cream composition of Example 1 may be reformulated to exclude isododecane. For example, a topical gel-cream composition according to the invention was made having the following ingredients, as shown in Table 3.

TABLE 3
Ingredient Listing for Example 3

| Ingredient | Primary Function | Weight % |
|---|---|---|
| Purified Water | Vehicle | Q.S. |
| Colloidal Oat Flour | Absorbant | 1.00 |
| Carbomer (Carbomer Ultrez 10 | Viscosity Increasing Agent | 0.40 |
| Polymer)¹ | | |
| Citric Acid Anhydrous USP | pH Adjusting Agent | 0.60 |
| Lidocaine USP | Pain Relieving Agent | 4.00 |
| Glycerine | Humectant | 14.00 |
| Sodium Polyacrylate² | Viscosity Increasing Agent | 0.75 |
| Isopropyl Palmitate³ | Emollient | 5.00 |
| Cetearyl Olivate; Sorbitan | Emollient | 0.50 |
| Olivate⁴ | | |
| Cetyl Alcohol⁵ | Emulsifier | 1.00 |
| Euphorbia Cerifera | Emollient | 2.00 |
| (Candelilla) Wax; | | |
| Hydrogenated | | |
| Vegetable Oil; | | |
| Vegetable Oil⁶ | | |
| Zingiber Officinale | Skin Conditioner | 0.20 |
| (Ginger) Root Extract | | |
| Caprylyl Glycol⁷ | Preservative System | 0.26 |
| | Component and | |
| | Skin Conditioner | |
| 1,2-Hexanediol; Caprylyl | Skin Conditioner | 0.50 |
| Glycol; Tropolone⁸ | | |
| Chlorphenesin⁹ | Preservative | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft VP from the UL Prospector Corporation
⁷Commercially available as Hydrolite CG from the Symrise Corporation
⁸Commercially available as Symdiol 68T from the Symrise Corporation
⁹Commercially available as Germazide C from the BASF Corporation

The following mixing procedure may be used for Example 3:
a) The purified water is added to a suitable vessel and the colloidal oat flower is added while mixing.
b) The citric acid and lidocaine USP are added while mixing and mixed until dissolved.
c) The carbomer is added while mixing slowly until all clumps are dissolved.
d) The mixture is heated until 75-80°° C.
e) The caprylyl glycol and chlorphenesin are added while mixing and mixed until uniform.
f) The sodium polyacrylate is then added slowly while mixing at 75-80° C. until uniform.
g) The oil phase ingredients are then added while mixing (7% of the glycerin, isopropyl palmitate, cetearyl olivate; sorbitan olivate, and cetyl alcohol).
h) The mixture is mixed for 10 minutes and then homogenized for 3 minutes at 5000 RPM.
i) The mixture is cooled to 25° C. and additional 7% of glycerin is added and mixed.
j) The zingiber officinale root extract, 1,2-Hexanediol; Caprylyl Glycol; Tropolone are added and mixed until homogeneous.
k) At or below 35° C., the pH is adjusted to 6.5 using Sodium Hydroxide Solution or Citric Acid Solution and mixed.
l) The batch is brought to volume with water to 95-100% of theoretical batch weight.

Example 4: Topical Cream without Isododecane

According to an example the topical cream composition of Example 2 may be reformulated to exclude isododecane. For example, the topical gel cream composition according to the invention was made having the following ingredients, as shown in Table 4.

TABLE 4
Ingredient Listing for Example 4

| | | Weight |
|---|---|---|
| Ingredient | Primary Function | % |
| Purified Water | Vehicle | Q.S. |
| Colloidal Oat Flour | Absorbant | 1.00 |
| Carbomer (Carbomer Ultrez 10 | Viscosity Increasing | 0.40 |
| Polymer)¹ | Agent | |
| Citric Acid Anhydrous | pH Adjusting Agent | 0.60 |
| Lidocaine USP | Pain Relieving Agent | 4.00 |
| Glycerine | Humectant | 12.00 |
| Sodium Polyacrylate² | Viscosity Increasing | 0.60 |
| | Agent | |
| Isopropyl Palmitate³ | Emollient | 5.00 |
| Cetearyl Olivate; Sorbitan Olivate⁴ | Emollient | 0.50 |
| Cetyl Alcohol⁵ | Emulsifier | 3.00 |
| Euphorbia Cerifera (Candelilla) | Emollient | 3.00 |
| Wax; Hydrogenated Vegetable Oil; | | |
| Vegetable Oil⁶ | | |
| Fragrance | Fragrance | 0.50 |
| Acrylates Copolymer⁷ | Viscosity Increasing | 2.00 |
| | Agent | |
| Caprylyl Glycol⁸ | Preservative System | 0.26 |
| | Component and | |
| | Skin Conditioner | |
| 1,2-Hexanediol; Caprylyl Glycol; | Skin Conditioner | 0.50 |
| Tropolone⁹ | | |
| Chlorphenesin¹⁰ | Preservative | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft BP from the UL Prospector Corporation
⁷Commercially available as Carbopol Aqua SF-1 Polymer from the Lubrizol Corporation
⁸Commercially available as Hydrolite CG from the Symrise Corporation
⁹Commercially available as Symdiol 68T from the Symrise Corporation
¹⁰Commercially available as Germazide C from the BASF Corporation

The following mixing procedure may be used to prepare Example 4:
a) The purified water is added to a suitable vessel and the colloidal oat flower is added while mixing.
b) The citric acid and lidocaine USP are added while mixing and mixed until dissolved.
c) The carbomer is added while mixing slowly until all clumps are dissolved.
d) The mixture is heated until 75-80°° C.
e) The caprylyl glycol and chlorphenesin are added while mixing and mixed until uniform.
f) The sodium polyacrylate is then added slowly while mixing at 75-80° C. until uniform.
g) The oil phase ingredients are then added while mixing (7% of glycerin, isopropyl palmitate, cetearyl olivate; sorbitan olivate, and cetyl alcohol).
h) The mixture is mixed for 10 minutes and then homogenized for 3 minutes at 5000 RPM.
i) The mixture is cooled to 25° C. and the fragrance, 1,2-hexanediol; caprylyl glycol; tropolone are added and mixed until homogeneous.
j) At or below 35° C., the pH is adjusted to 6.5 using sodium hydroxide solution or citric acid solution and mixed.
k) In a different vessel 5% glycerin and acrylates Copolymer are added and mixed until uniform to create the acrylates copolymer premix.
l) The acrylates copolymer premix is added to the mixture from Step 11 and mixed until homogenous.

### Human Use Study

### Study Protocol and Objective

A home use test (abbreviated as “HUT”) was conducted where 253 consumers (also referred to as “subjects,” “patients” or “users”) self-administered the formulas in Examples 1 and 2 (also referred to in this example as “formula(s)” or “product(s)”) as needed for a period of two weeks. These consumers were between 30 and 55 years old, who regularly or occasionally experience minor aches and pains of the muscles or joints, and who had a range of skin tones and types with a sub-set having self-reported sensitive skin, dry skin, and/or eczema.

The consumers were instructed to use the product within the first two days of receiving it and then at least once a week as needed after that, over the following two weeks. The consumers were further instructed to abstain from using other pain-relieving treatments during the two-week period. The consumers provided feedback via an online survey at three time points: 1) after first use, 2) after one week of use, and 3) after two weeks of use.

Example 1 may be referred to as a “soothing gel-cream” incorporating, for example, ginger and Example 2 may be referred to as a “calming cream” incorporating a suitable calming fragrance, for example, lavender.

The objective of the human use study was to generate support for potential consumer perceptual claims centered around a consumers' experience with the product. For example, an objective was to understand the consumers' experience with regard to product absorption, discreetness and feel on the skin.

With regard to feel, consumers provided feedback on whether the product was perceived to be gentle and whether the product made their skin feel moisturized and/or silky.

A “test statement” may be a question that is answered or a statement that is agreed or disagreed with at least once by the consumer during the duration of the HUT. According to an example, the following test statements may relate to the consumers' experience with regard to product absorption: is fast absorbing, instantly absorbs into the skin, is non-greasy, and/or doesn't leave my skin shiny. According to an example the following test statements may relate to the consumers' experience with regard to “discreetness: ” is lightweight, does not have a harsh smell/odor, has no unpleasant smell/odor, is non-sticky, is non-greasy, doesn't leave my skin shiny, has a pleasant smell. According to an example, the following test statements may relate to the consumers' experience with regard to product feel: is lightweight, is non-sticky, is non-greasy, instantly moisturizes, leaves my skin feeling silky, instantly cools, feels like it is working right away and/or instantly numbs. The test statements of instantly cools, feels like it is working right away and/or instantly numbs may indicate pain relief.

### Summary of the Results

The top line data is presented in Table 5, and shows the reactions as described by consumer, collated as a percentage of users in agreement with the test statement. Total results, as well as results for each of Example 1 and Example 2 are shown.

TABLE 5
Human Use Study Results

| | Percentage (%) of Consumers in Agreement | | |
|---|---|---|---|
| | Total | Example 1 | Example 2 |
| Test Statement | (n = 249)_{—} | (n = 127) | (n = 122) |
| is easy to apply | 98 | 98 | 98 |
| is lightweight | 95 | 95 | 94 |
| does not have a | 94 | 94 | 93 |
| harsh smell/odor | | | |
| has no unpleasant | 92 | 94 | 90 |
| smell/odor | | | |
| is non-sticky | 92 | 89 | 94 |
| is fast absorbing | 90 | 90 | 91 |
| instantly absorbs | 87 | 87 | 88 |
| into the skin | | | |
| is non-greasy | 87 | 87 | 87 |
| instantly moisturizes | 83 | 79 | 88 |
| doesn't leave my | 82 | 86 | 78 |
| skin shiny | | | |
| has a pleasant smell | 79 | N/A | 79 |
| leaves my skin | 73 | 72 | 73 |
| feeling silky | | | |
| instantly cools | 63 | 61 | 64 |
| feels like it's | 62 | 61 | 64 |
| working right away | | | |
| instantly numbs | 53 | 50 | 55 |

* Not Applicable: Formula was fragrance free.

Over 90% of consumers found both Examples 1 and 2 to be easy to apply, light weight, and fast absorbing. Over 90% of consumers also found Examples 1 and 2 to be without a harsh and unpleasant smell/odor. Example 1 appeared to have better smell perception than Example 2.

Greater than 60% of consumers also agreed that Examples 1 and 2 were non-sticky, absorbed instantly into the skin, were non-greasy, instantly moisturized, did not leave skin shiny, had a pleasant smell, left skin feeling silky, instantly cooled and felt like they worked right away. Example 2 appeared to be more non-sticky than Example 1. Example 2 appeared to provide more instant moisturization than Example 1. Example 1 appeared to leave skin less shiny than Example 2. Example 2 appeared to instantly cool and felt like it worked right away more so than Example 1.

Approximately 50% of users agreed that both formulas instantly numbed the skin, however, it appeared that Example 2 was more numbing than Example 1.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A topical composition comprising: a topical analgesic and a volatile emollient, wherein the topical composition is substantially free of volatile alcohol.

2. The topical composition of claim 1, wherein the topical analgesic is in an amount of 0.5% to 8% by weight.

3. The topical composition of claim 1, wherein the topical analgesic is one or more of lidocaine or benzocaine.

4. The topical composition of claim 3, wherein the topical analgesic is lidocaine.

5. The topical composition of claim 4, wherein the lidocaine is an amount of about 4% by weight.

6. The topical composition of claim 1, wherein the volatile emollient is in an amount of 1% to 12% by weight.

7. The topical composition of claim 1, wherein the volatile emollient is an amount between about 4% to about 8% by weight.

8. The topical composition of claim 1, wherein the volatile emollient provides a topical cooling sensation.

9. The topical composition of claim 1, wherein the volatile emollient comprises isododecane.

10. The topical composition of claim 1, further comprising an absorbant.

11. The topical composition of claim 10, wherein the absorbant is in an amount of 0.1% to 5% by weight.

12. The topical composition of claim 10, wherein the absorbant comprises a processed oat ingredient.

13. The topical composition of claim 12, wherein the processed oat ingredient comprises colloidal oat flour.

14. The topical composition of claim 13, wherein the colloidal oat flour is in an amount of about 1% by weight.

15. The topical composition of claim 1, wherein the topical composition is substantially free of menthol and menthol lactate.

16. A method of relieving pain in a user with sensitive skin, comprising applying the topical composition of claim 1 to an exterior skin portion of the user's body in need of pain treatment.

17. A method of relieving pain in a user with sensitive skin, comprising topically applying to an exterior skin portion of a user's body in need of pain treatment a composition comprising lidocaine and isododecane, wherein the composition is substantially free of volatile alcohol.

18. The method of claim 17, wherein the lidocaine is in an amount of 0.5% to 8% by weight, and wherein the isododecane is in an amount of 1% to 12% by weight.

19. The method of claim 17, wherein the composition further comprises a processed oat ingredient.

20. The method of claim 19, wherein the processed oat ingredient is an amount of 0.1% to 5% by weight.

21-33. (canceled)
